(19) United States
(12) Patent Application Publication
(10) Pub. No.: US 2025/0082502 A1
(43) Pub. Date: Mar. 13, 2025

(54) TAPERED OPTICAL FIBERS FOR SURGICAL INSTRUMENTATION

(71) Applicant: Alcon Inc., Fribourg (CH)

(72) Inventors: John Morgan BOURNE, Irvine, CA (US); Gisele MAXWELL, Temecula, CA (US); Mikhail OVCHINNIKOV, Dana Point, CA (US); Adela APOSTOL, Lake Forest, CA (US)

(21) Appl. No.: 18/819,507

(22) Filed: Aug. 29, 2024

Related U.S. Application Data
(60) Provisional application No. 63/581,700, filed on Sep. 11, 2023.

(51) Int. Cl. A61F 9/008; A61B 17/00; A61B 18/00; A61B 18/22

(52) U.S. Cl. CPC A61F 9/008; A61B 2017/00199; A61B 2018/0091; A61B 2018/2211; A61B 2018/2222; A61B 2218/007

(57) ABSTRACT

Embodiments disclosed herein relate to devices and systems for use in ophthalmic procedures. In some embodiments, a handpiece device for use with a surgical system is provided. The handpiece device includes a housing, a cable assembly, and a tapered optical fiber. The cable assembly is disposed through a distal end of the housing and optically coupled to the tapered optical fiber. The tapered optical fiber has a diameter that decreases between a proximal end of the tapered optical fiber and a distal end thereof. In some embodiments, the tapered optical fiber comprises a single-crystal fiber. In further embodiments, the tapered optical fiber comprises a tapered sapphire core fiber.

### CROSS-REFERENCE TO RELATED APPLICATIONS

This application claims priority to U.S. Provisional Application No. 63/581,700, filed on Sep. 11, 2023, which is hereby incorporated by reference in its entirety.

### BACKGROUND

In a wide variety of ophthalmic procedures, laser light is used to perform surgery and/or to treat patient anatomy. For example, in laser photoemulsification, a probe of a microsurgical instrument projects a beam of laser light to emulsify and ablate the crystalline lens of an eye for cataract removal. In a vitrectomy procedure, a surgeon inserts a microsurgical instrument, such as a vitrectomy probe, through one or more incisions made in the eye to cut and remove the vitreous body from within. Microsurgical instruments utilized for such procedures can use laser light transmitted from a laser system through one or more optical fibers that distally terminate in the probe to perform one or more functions of the procedure. In some instances, such as in the case of the vitrectomy procedure, the vitrectomy probe cuts the vitreous body with laser light delivered from the laser system and then subsequently aspirates the cut biological material to remove it.

### SUMMARY

The present disclosure generally relates to optical fibers, and more specifically, to components for energy delivery in surgical laser systems. In some embodiments, a handpiece device for use in ophthalmic surgical procedures is provided. The handpiece device includes a housing and a cable assembly disposed within the housing with the cable assembly extending from a supply port at a proximal end of the housing. The cable assembly comprises an optical fiber optically connected to a tapered optical fiber disposed in the housing and having a diameter that decreases between a proximal end of the tapered optical fiber and a distal end of the tapered optical fiber. The proximal end of the tapered optical fiber is optically coupled to a distal end of the optical fiber in the cable assembly.

In some embodiments, a cable assembly for a surgical system is provided. The cable assembly comprises an optical fiber having a proximal end and a distal end, a first tapered optical fiber having a proximal end and a distal end, and a second tapered optical fiber. The distal end of the first tapered optical fiber is optically coupled to the proximal end of the optical fiber, wherein the first tapered optical fiber comprises a first diameter that decreases from the proximal end of the first tapered optical fiber to the distal end of the first tapered optical fiber, and a diameter of the distal end of the first tapered optical fiber is less than a diameter of the proximal end of the optical fiber. The second tapered optical fiber includes a proximal end optically coupled to the distal end of the optical fiber.

In other embodiments, a surgical system is provided. The surgical system includes a surgical console having a laser system and a handpiece device in connection with the laser system. The handpiece device includes a housing and a cable assembly disposed within the housing and extending from a supply port at a proximal end of the housing wherein the cable assembly comprises an optical fiber having a proximal end and a distal end. The handpiece device also includes a tapered optical fiber disposed in the housing and having a diameter that decreases between a proximal end of the tapered optical fiber and a distal end of the tapered optical fiber, wherein the proximal end of the optical fiber is optically coupled with the laser system, and the distal end of the optical fiber is optically coupled to the proximal end of the tapered optical fiber.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a side view of an exemplary surgical system for use during an ophthalmic surgical procedure, according to certain embodiments of the present disclosure.

FIGS. 1B illustrates a perspective view of an exemplary handpiece device that may be implemented in the surgical system of FIG. 1A, according to certain embodiments of the present disclosure.

FIG. 1C illustrates a longitudinal cross-sectional view of an exemplary handpiece device that may be implemented in the surgical system of FIG. 1A, according to certain embodiments of the present disclosure.

FIG. 2A illustrates a plan view of a distal end of the handpiece device of FIGS. 1B and 1C, according to certain embodiments of the present disclosure.

FIGS. 2B-2D illustrate longitudinal cross-sectional views of a distal end of the handpiece device of FIGS. 1B and 1C, according to certain embodiments of the present disclosure.

FIG. 3 illustrates a longitudinal schematic view of an exemplary cable assembly that may be implemented in the surgical system in FIG. 1A, according to certain embodiments of the present disclosure.

FIG. 4 illustrates a longitudinal schematic view of another exemplary cable assembly that may be implemented in the surgical system in FIG. 1A, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical console.

As used herein, the term “about” may refer to a +/−10% variation from the nominal value. It is to be understood that such a variation can be included in any value provided herein.

Embodiments disclosed herein generally relate to optical fibers for use with surgical systems, and more particularly, tapered optical fibers for use with surgical laser systems and microsurgical instruments connected thereto. The methods, systems, and apparatus described herein may be utilized in combination with any suitable surgical instruments for ophthalmic procedures having light emitting functionality (e.g., laser light, illumination light, etc.), such as those described below.

In surgical systems where laser light is used in ophthalmic surgery, such systems typically rely on delivery of the laser energy from a laser source to the patient via one or more optical cable assemblies. In such surgical systems, the optical cable assemblies may include one or more optical fibers extending between the laser source and a microsurgical instrument. When in use, laser light delivered by the one or more optical fibers can be projected from a probe of the microsurgical instrument for the surgical procedure. Various properties of the one or more optical fibers can affect the delivery of laser light and the implementation of such optical fibers in the microsurgical instrument. For example, both the diameter and the materials of the one or more optical fibers can affect the efficiency of the optical fiber in the transmission of laser light, as well as the flexibility and durability of the corresponding optical cable assembly thereof.

The microsurgical instruments typically utilized during such surgical procedures also provide aspiration through the probe during the surgical procedure. The diameter of the optical fiber can therefore affect the implementation of the optical fiber and/or optical cable assembly with the microsurgical instrument, such as the extension of the optical fiber through the probe of the instrument and the maintaining of adequate space in the probe for aspiration. However, as surgical instruments having probes with smaller gauges are increasingly implemented for ophthalmic procedures (e.g., due to the potential for improved safety and shorter healing periods), optical fibers with correspondingly smaller diameters are needed to deliver laser light to the probe as well as to continue providing aspiration through the probe of the microsurgical instruments. As such, in order for the microsurgical instrument to provide both laser and aspirating functions, a balance between efficient transmission of laser light by the optical fiber and the maintaining of sufficient space in the probe (e.g., around the optical fiber) for aspiration is key.

In certain existing surgical systems, the optical cable assembly for delivering laser light includes an optical fiber with a 200 μm (micrometer) core. In such surgical systems, decreasing the diameter of some optical fibers (e.g., decreasing a core of an optical fiber below 200 μm) to accommodate for smaller gauge probes in the microsurgical instrument connected thereto may result in unacceptable optical transmission losses. For example, some optical fibers exhibit optical loss when used to deliver laser light across greater distances (e.g., such as distances greater than about 50 mm (millimeters), such as distances about 2 meters). Furthermore, as the diameter of an optical fiber is decreased, the durability of the optical fiber may also be reduced, thereby shortening the useful lifetime of such components. On the other hand, optical fibers with 200 μm (or greater) cores suitable for facilitating transmission across greater distances from the laser source can be relatively stiff and are likely to hamper movement of the microsurgical instrument connected thereto. In such instances, the one or more optical fibers with 200 μ(or greater) cores may in turn present ergonomic issues for the end user or surgeon manipulating the microsurgical instrument.

Embodiments of the present disclosure provide a system for laser light transmission via one or more optical fibers that reduce optical loss and the likelihood of cable assembly failure when delivering laser light from the laser source to the probe of the microsurgical instrument. As discussed below in further detail, various techniques described herein implement a tapered optical fiber that enables laser light to be projected from the probe by a distal end of an optical fiber with a reduced diameter. For example, the tapered optical fiber can be used to transmit laser light through an optical fiber or a portion of an optical fiber with a smaller diameter and disposed in the probe of the microsurgical instrument to enable aspiration and/or improve flow through the probe. The tapered optical fiber can also be used to decrease the diameter of a portion of an optical fiber disposed near the microsurgical handpiece device to increase the flexibility of the optical fiber at the specific portion thereof.

The one or more optical fibers of the optical cable assemblies extending between the laser source and the probe of the microsurgical instrument may, in some aspects, include two or more optical fibers that are coupled together to provide specific properties to different portions of the optical cable assembly (e.g., an optical fiber having greater flexibility coupled to an optical fiber having better transmission). When laser light is transmitted through the cores of the two or more optical fibers, the cores of adjacent optical fibers are aligned when coupling the optical fibers together to facilitate the transmission of laser light between the respective cores of the coupled optical fibers. However, due to the tolerances of the diameter and centricity of the core of each such coupled optical fiber, a portion of the laser light transmitted between the coupled ends of adjacent optical fibers (e.g., a distal end of one optical fiber and a proximal end of an adjacent optical fiber at a coupling interface) may be partially delivered to the adhesive surrounding the core of the optical fiber. In such instances, the errant energy from the laser light can heat the adhesive of the optical fiber receiving the laser light and ultimately damage and/or cause the optical cable assembly to fail.

Embodiments of the present disclosure include techniques for implementing the tapered optical fibers at the coupling interfaces between adjacent optical fibers of the optical cable assembly. A tapered optical fiber disposed at the coupling interface between adjacent coupled optical fibers can assist in concentrating the delivery of the laser light on a smaller area aligned with the core of the optical fiber receiving the laser light. By coupling adjacent optical fibers with the tapered optical fiber, the techniques described herein decrease the likelihood of a partial alignment and corresponding delivery of errant laser energy to the adhesive of coupled optical fibers and/or the optical cable assembly.

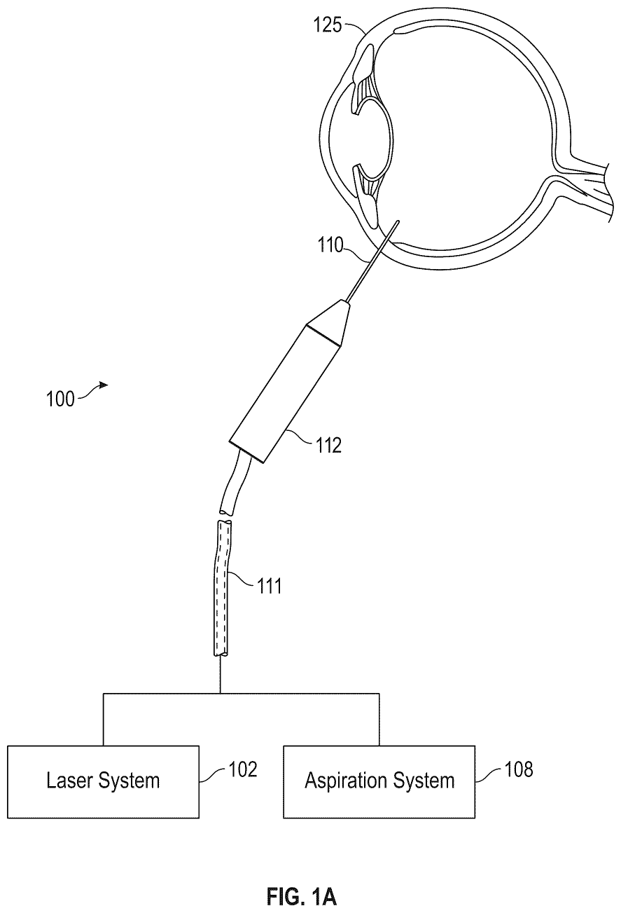

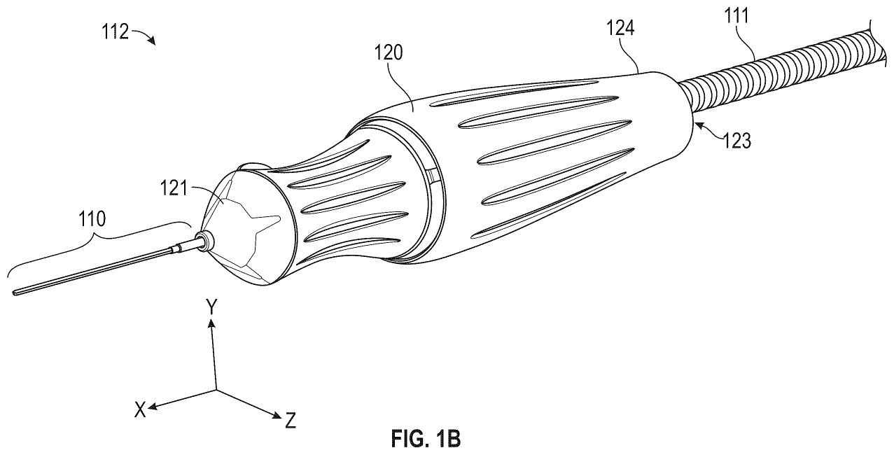

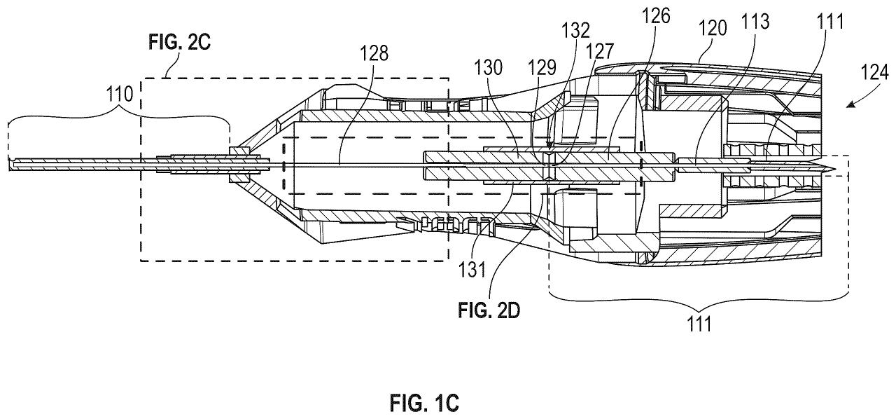

FIG. 1A illustrates an exemplary surgical system 100 performing laser-assisted ophthalmic surgical procedures, according to certain embodiments of the present disclosure. In certain embodiments, the surgical system 100 may include a surgical console similar to ophthalmic surgical consoles that have been known and used, such as the CONSTELLATION® Vision System available from Alcon Laboratories, Inc. (Fort Worth, Texas) or the CENTURION® Vision System available from Alcon Laboratories, Inc. (Fort Worth, Texas), or any other ophthalmic surgical console suitable for use with the principles described herein.

As shown, the surgical system 100 includes a laser system 102 having one or more laser sources connected to a handpiece device 112 by a cable assembly 111. The surgical system 100 may also include an aspiration system 108 having a vacuum source in fluid communication with the handpiece device 112 for providing aspiration through the handpiece device 112. In certain embodiments, the laser system 102 and/or the aspiration system 108 may be part of or incorporated into the surgical console described above. The aspiration system 108 may also be connected to the handpiece device 112 by the cable assembly 111. In some embodiments, the cable assembly 111 may comprise one or more cables connected together. In some embodiments, the cable assembly 111 includes one or more optical fibers for delivering laser light from the laser system 102 to the handpiece device 112. In some embodiments, the cable assembly 111 may further include a vacuum line for providing aspiration to the handpiece device 112.

In certain embodiments, the handpiece device 112 may include a vitrectomy probe or the like for laser-assisted procedures such as for cutting of vitreous fiber materials in the eye of a patient. FIG. 1A shows a probe 110 of the handpiece device 112 inserted into a vitreous of a patient eye 125 for performing an ophthalmic surgical procedure. As the probe 110 is moved through the vitreous, laser light is emitted by one or more optical fibers of the cable assembly 111 within the probe 110. The user, such as a surgeon, may toggle the laser light between on and off positions using a switch on the handpiece device 112, a foot pedal connected to the surgical system 100, or other means. The switch and/or the foot pedal may also be configured to control aspiration by the handpiece device 112, such as for the removal of severed vitreous body from the intraocular space of the eye 125 during a vitreoretinal procedure. In other embodiments, the handpiece device 112 may be configured such that the laser light delivered by the one or more optical fibers of the cable assembly 111 to the handpiece device 112 is used to perform other laser-assisted ophthalmic procedures, such as ablation (e.g., for treating the anterior segment of the eye 125) and phacoemulsification. In other words, the handpiece device 112 shown in FIG. 1A is merely exemplary and for illustration purposes only. In particular, although the handpiece device 112 is illustrative of a vitrectomy handpiece device with probe 110 as entering the vitreous, the embodiments herein are similarly applicable to handpiece devices used for other surgical procedures, such as photocoagulation, photo and/or phacoemulsification, etc.

FIG. 1B illustrates a perspective view of an exemplary handpiece device 112 that may be implemented in the surgical system 100 described above, according to certain embodiments of the present disclosure. The handpiece device 112 includes a probe 110 and a housing 120. The probe 110 is partially and longitudinally disposed through a distal end 121 of the housing 120 and may be directly or indirectly attached thereto within an interior chamber of the housing 120.

The housing 120 further includes one or more supply ports 123 (e.g., one supply port 123 is depicted in FIG. 1B) at a proximal end 124 thereof for one or more supply lines to be routed into an interior chamber of the housing 120. For example, the supply port 123 may provide a connection between the housing 120 and the cable assembly 111 disposed through the proximal end 124 of the housing 120. The supply port 123 may also provide a connection to the aspiration system 108 for aspiration by the handpiece device 112. In some embodiments, the handpiece device 112 may be sterilized and used in more than one surgical procedure, or the handpiece device 112 may be a single-use device. In the instance in which the handpiece device 112 is a single-use device, the cable assembly 111 is connected and disconnected from the housing 120 through the supply port 123 before and after each surgical procedure.

In some embodiments, the housing 120 may have an outer surface configured to be held by a user, such as a surgeon. For example, the housing 120 may be ergonomically contoured to substantially fit the hand of the user. As shown in FIG. 1B, the outer surface may be textured or have one or more gripping features formed thereon, such as one or more grooves and/or ridges. The housing 120 may be made from any materials commonly used for such instruments and suitable for ophthalmic surgery. For example, the housing 120 may be formed of a lightweight aluminum, a polymer, or other suitable material.

A distal end of the cable assembly 111 is coupled to the handpiece device 112. In some embodiments, a proximal end of the cable assembly 111 may in turn be coupled to the laser system 102 with a light source (e.g., laser light source, illumination light source, etc.). The one or more optical fibers of the cable assembly 111 are configured to deliver laser light provided by the laser source to the handpiece device 112. In some embodiments, the proximal end of the cable assembly 111 may also connect to a vacuum source, such as the vacuum source of the aspiration system 108, for providing aspiration to the handpiece device 112.

In certain embodiments, the cable assembly 111 has a length between about 1 meter and about 3 meters, such as about 2 meters, but may have a larger or smaller length in some embodiments. In some embodiments, the cable assembly 111 includes one or more optical fibers 113. In some embodiments, the optical fiber 113 comprises a core and a cladding layer circumferentially surrounding the core. Generally, the one or more optical fibers 113 of the cable assembly 111 may be disposed in a cable coating to further support and protect each of the one or more optical fibers 113.

In certain embodiments, the core of the optical fiber 113 may include any transparent material, such as fused silica or glass. In some embodiments, the optical fiber 113 may include a germanate glass (e.g., GeO₂), a fluoride glass (e.g., ZBLAN, aluminum fluoride, etc.), or other material. In some embodiments, the core of the optical fiber 113 may be doped. For example, the core of the optical fiber 113 may be germanium-doped silica. Doping the core of the optical fiber 113 with germanium or a similar dopant may increase the refractive index of the core of the optical fiber 113 compared to that of the cladding, hence enabling laser light guiding properties within the core of the optical fiber 113. The cladding may also include a transparent material, such as fused silica or glass. In some embodiments, the cladding is doped in addition to or instead of doping the core. For example, the cladding, which may include fused silica, is doped with a dopant that reduces the refractive index of the cladding relative to that of the core. Example dopants include fluorine (F), chlorine (Cl), boron (B), or the like. The cladding, when doped, has a lower refractive index than the core, thus enabling light guiding properties within the core.

In certain embodiments, the optical fiber 113 comprises a hollow core fiber capable of guiding light through a hollow region/air core within the fiber. In some embodiments, the optical fiber 113 may be a hollow core fiber configured with a silver reflective coating, a hollow core photonics crystal fiber, or an anti-resonant hollow core fiber.

FIG. 1C illustrates a longitudinal cross-sectional view of the handpiece device 112 with the optical fiber 113 of the cable assembly 111 coupled to a tapered optical fiber 128 disposed within the housing 120. As discussed above, a distal end 127 of the optical fiber 113 extends into the housing 120 from the proximal end 124 of the housing 120. In certain embodiments, the core of the optical fiber 113 may have a diameter between about 170 μm and about 280 μm, such as between about 200 μm and about 250 μm, such as about 200 μm, but may have a larger or smaller diameter in some embodiments. In certain embodiments, the core and cladding of the optical fiber 113 may have a combined diameter between about 180 μm and about 400 μm, such as between about 180 μm and about 250 μm, or between about 250 μm and about 400 μm, or between about 200 μm and about 300 μm, such as about 230 μm, but may have a larger or smaller combined diameter in some embodiments.

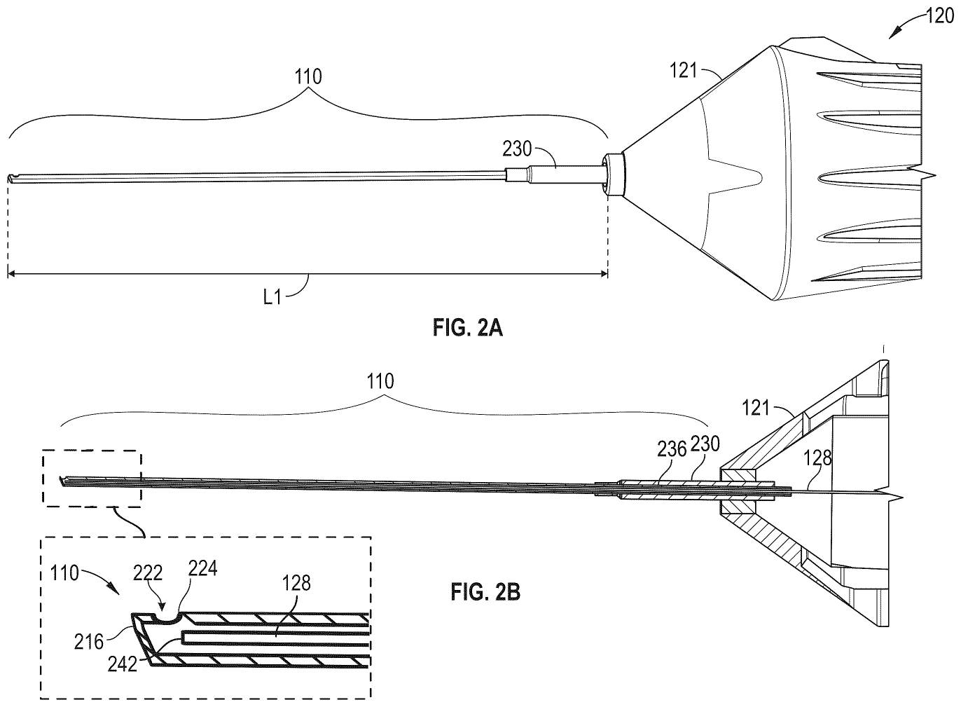

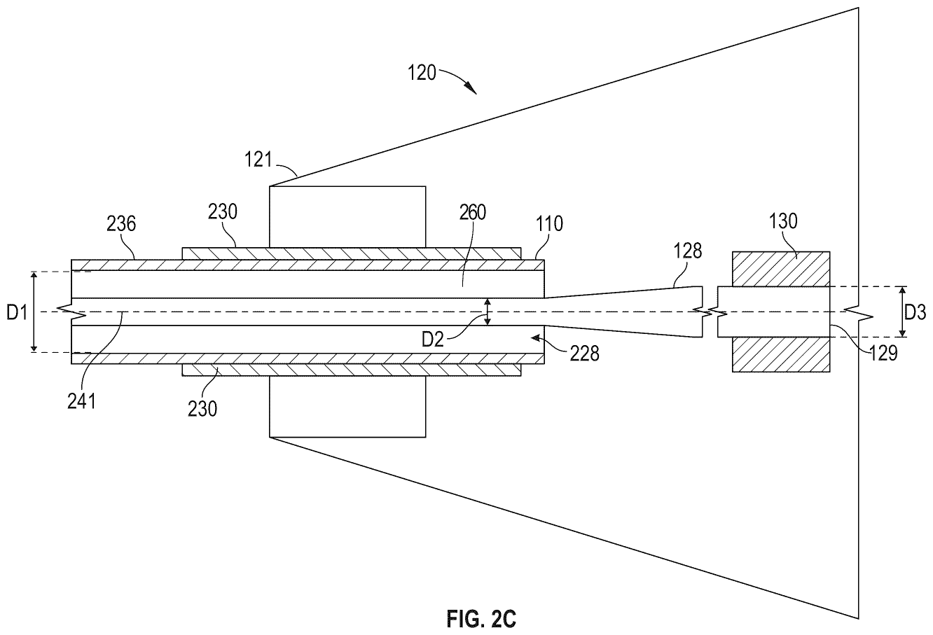

In some embodiments, the optical fiber 113 is coupled to the tapered optical fiber 128 to enable transmission of laser light from the housing 120 through the probe 110 of the handpiece device 112. As shown in FIG. 1C (and FIG. 2D discussed further below and showing an enlarged detailed view of a selected portion of FIG. 1C), a proximal end 129 of the tapered optical fiber 128 connects with the distal end 127 of the optical fiber 113 at a coupling interface 132 in the housing 120. As described in more detail below (e.g., with reference to FIGS. 2C and 2D), a distal portion of the tapered optical fiber 128 includes a diameter smaller than the diameter of the core and/or cladding of the optical fiber 113, thereby enabling the partial extension of the tapered optical fiber 128 into the probe 110. As discussed above, in some embodiments, the tapered optical fiber 128 partially disposed within the probe 110 is configured to project a laser beam inside the probe 110 from a distal tip thereof (e.g., for laser assisted ophthalmic functions such as severing vitreous material).

In the housing 120, the handpiece device 112 further includes a ferrule 126 disposed at and around the distal end 127 of the optical fiber 113 adjacent to a ferrule 130 disposed at the proximal end 129 of the tapered optical fiber 128. In some embodiments, the distal end 127 of the optical fiber 113 and the ferrule 126 disposed thereon are in optical contact (e.g., butt coupled) with the proximal end 129 of the tapered optical fiber 128 and the ferrule 130 thereof such that laser light may be transmitted from the optical fiber 113 of the cable assembly 111 to the tapered optical fiber 128.

In some embodiments, a slight air gap may form at the coupling interface 132 between the butt coupled ends of the optical fiber 113 and the tapered optical fiber 128. In certain embodiments, portions of the optical fiber 113, the tapered optical fiber 128 and the ferrules 126, 130 at the coupling interface 132 are further disposed within a sleeve 131 to maintain the optical connection between the optical fiber 113 and the tapered optical fiber 128. In some embodiments, the sleeve 131 includes a cylindrical tube configured to clamp down on the coupling ends of the ferrules 126, 130 and the ends of the optical fiber 113 and the tapered optical fiber 128 at the coupling interface 132. The ferrules 126, 130 and the sleeve 131 assist in protecting and aligning the coupling ends of the optical fiber 113 and the tapered optical fiber 128 to reduce transmission loss at the coupling interface 132. In some embodiments, the ferrules 126, 130 may be or include a metallic tube, a ceramic tube, a sapphire tube, or other material.

As mentioned above, in some embodiments, the handpiece device 112 is for single-use application, and a user may therefore connect a new handpiece device 112 with the cable assembly 111 prior to each surgical procedure. In some embodiments, the distal end 127 of the optical fiber 113 and the ferrule 126 disposed thereon are configured to removably connect to the proximal end 129 of the tapered optical fiber 128 and the ferrule 130 such that the distal end 127 and the ferrule 126 may be separated and extracted from the housing 120. To disconnect the cable assembly 111 from the handpiece device 112, the user withdraws the distal end 127 of the optical fiber 113 and the ferrule 126 from the sleeve 131 and the proximal end 124 of the housing 120. To connect the cable assembly 111 to a new handpiece device 112, the user subsequently inserts the distal end of the optical fiber 113 and the ferrule 126 disposed thereon into the sleeve 131 of the new handpiece device 112 to couple the optical fiber 113 and the tapered optical fiber 128 in the new handpiece device 112 together.

FIG. 2A illustrates a plan view of the probe 110 and the distal end 121 of the housing 120. As shown, the probe 110 may be an elongated laser cutting member that may be inserted into an eye (e.g., through an insertion probe, for performing laser-assisted ophthalmic procedures, which may be aspirating or non-aspirating). The probe 110 may thus be formed of materials suitable for minimally invasive ophthalmic surgeries. In some embodiments, the probe 110 includes one or more sections that are formed of materials configured to transmit laser light, visible light, ultraviolet light, infrared light, or any other type of light. For example, the probe 110 may include one or more sections formed of a translucent or transparent material, such as a plastic and/or polymeric material. The probe 110 may further include one or more sections formed of more conventional surgical-grade materials, such as stainless steel and/or aluminum.

In certain embodiments, the probe 110 has a length L1 between about 15 mm and about 30 mm, but may have a larger or smaller length in some embodiments. In some embodiments, the handpiece device 112 further includes a stiffener 230 fixedly or slidably coupled to and substantially surrounding at least a portion of the probe 110. For example, the stiffener 230 may be slidably coupled to an exterior surface 236 (shown in FIGS. 2B-2C) of the probe 110 and may extend from and retract into the housing 120. The stiffener 230 may be adjustable relative to the probe 110, enabling a user to position the stiffener 230 at different points along the length L1 of the probe 110 exterior to the housing 120. Accordingly, a user may selectively adjust the level of stiffness of the probe 110 by re-positioning the stiffener 230 relative to the distal tip 216, thereby manipulating the amount of support provided to the probe 110 and stabilizing the handpiece device 112 during use thereof.

FIG. 2B illustrates a longitudinal cross-sectional view of the probe 110 and the distal end 121 of the housing 120 with the tapered optical fiber 128 disposed therein, according to certain embodiments of the present disclosure. FIG. 2C illustrates a close up partial longitudinal cross-sectional view of the extension of the tapered optical fiber 128 into the probe 110, according to certain embodiments of the present disclosure. FIG. 2D illustrates a longitudinal cross-sectional view of the coupling of the tapered optical fiber 128 and the optical fiber 113 at the coupling interface 132, according to certain embodiments of the present disclosure. FIGS. 2B-2D are described together herein for clarity.

As depicted in FIG. 2B, the probe 110 includes a lumen 260 and a port 222 near a distal tip 216. The lumen 260 may have an inner diameter D1 ranging between about 220 μm and about 500 μm. In some embodiments, the lumen 260 has a substantially circular cross section. However, a lumen 260 having a non-circular cross section (e.g., square or octagonal) is also contemplated. In some embodiments, the port 222 is sized and shaped to allow vitreous collagen fibers to enter the lumen 260 during vitrectomy. The vitreous collagen fibers may be aspirated into the lumen 260 through the port 222. In some embodiments, the distal end 242 of the tapered optical fiber 128 is disposed such that the tapered optical fiber 128 projects the laser beam across the port 222 for severing vitreous collagen fibers disposed within the lumen 260. In certain other embodiments, the port 222 may be disposed such that the distal end 242 of the tapered optical fiber 128 is configured to project the laser beam from the distal tip 216 of the probe 110.

As shown in FIG. 2C, the tapered optical fiber 128 may be rigidly suspended within the lumen 260 such that the tapered optical fiber 128 is separated from an interior sidewall of the probe 110 and is circumferentially surrounded by an annular gap 228. In some embodiments, the positioning of the tapered optical fiber 128 is maintained by an adhesive disposed around the tapered optical fiber 128 adjacent to a distal end of the ferrule 130, as shown in FIG. 2C. The annular gap 228 formed between the tapered optical fiber 128 and the interior sidewall of the probe 110 provides a coaxial path for aspiration of ablated, cut, and/or severed vitreous material through the probe 110. In some embodiments, the tapered optical fiber 128 may be centrally disposed within the lumen 260 such that a radial distance between the interior sidewall and the tapered optical fiber 128 is uniform along a circumference of the tapered optical fiber 128.

In certain embodiments, the tapered optical fiber 128 has a length between about 30 mm and about 50 mm, such as between about 35 mm and about 43 mm, such as about 40 mm, but may have a larger or smaller length in some embodiments. As shown in FIGS. 2A and 2B, the distal end 242 of the tapered optical fiber 128 may terminate at any point along the length L1 of the probe 110 to enable optimal severance of vitreous fibers as well as aspiration thereof. In some embodiments, the distal end 242 of the tapered optical fiber 128 terminates within the lumen 260 at a point distal to a proximal end 224 of the port 222. In certain other embodiments, the distal end 242 of the tapered optical fiber 128 terminates at a point within the lumen 260 substantially aligned with the proximal end 224 of the port 222. In still other embodiments, the distal end 242 of the tapered optical fiber 128 terminates within the lumen 260 at a point proximal to the proximal end 224 of the port 222.

As mentioned above, the tapered optical fiber 128 includes a tapered diameter such that a diameter D2 of a portion of the tapered optical fiber 128 disposed in the probe 110 is smaller than a diameter D3 of the portion of the tapered optical fiber 128 disposed in the housing 120, and hence may also be smaller than a diameter of the distal end 127 of the optical fiber 113. Decreasing the diameter of the portion of the tapered optical fiber 128 in the probe 110 correspondingly increases the annular gap 228 circumferentially surrounding the tapered optical fiber 128 in the probe 110 and/or maintains the annular gap 228 in a smaller gauged the probe 110. Increasing the size of the annular gap 228 in the probe 110 enables sufficient flow through the lumen 260, which in turn is beneficial for aspirating fluid or vitreous material severed by the laser beam projected from the tapered optical fiber 128. Having a sufficient annular gap 228 in the probe 110 is also advantageous for preventing and/or reducing the chances of blockages occurring in the probe 110 during such aspiration.

In some embodiments, the diameter D3 of the proximal end 129 of the tapered optical fiber 128 at the coupling interface 132 may substantially match the diameter of the distal end 127 of the optical fiber 113, such as being between about 180 μm and about 300 μm, such as between about 200 μm and about 250 μm, such as about 200 μm, but may have a larger or smaller diameter in some embodiments. From the proximal end 129, the diameter of the tapered optical fiber 128 tapers or decreases as the tapered optical fiber 128 approaches the probe 110. In certain embodiments, the diameter D2 for portions of the tapered optical fiber 128 disposed in the lumen 260 of the probe 110 may be between about 50 μm and about 150 μm, such as between about 80 μm and 120 μm, such as about 100 μm, but may have a larger or smaller diameter in some embodiments depending on the size of the lumen 260.

In some embodiments, the tapered optical fiber 128 includes a single-crystal optical fiber at least partially disposed within a cable coating to protect the tapered optical fiber 128 from surface contamination. In some embodiments, the single-crystal optical fiber of the tapered optical fiber 128 is a single-crystal sapphire optical fiber made from α-Al₂O₃. In other embodiments, the single-crystal optical fiber of the tapered optical fiber 128 may be made of Ti:Sapphire, Y₃Al₅O₁₂ (YAG), Ho:YAG, Yb:YAG, Nd:YAG, Er:YAG, Ce:YAG, Cr:YAG, or ZrF₄—BaF₂—LaF₃—AlF₃—NaF (ZBLAN). However, any material capable of propagating a laser light 241 and wavelength thereof as discussed further below may be used and are also contemplated. In such embodiments, in contrast to optical fibers having both a core and a cladding layer, single-crystal optical fibers lack a cladding layer and may therefore generally have a smaller diameter and/or may be thinner than optical fibers that have a cladding layer, while still being able to propagate laser light with similar or better efficiency.

Compared with amorphous silica or germanium-oxide fibers, single-crystal optical fibers offer higher thermal conductivity which in turn provides the advantages of propagating the laser light 241 with higher efficiency. In addition to providing an increased annular gap 228 in the lumen 260, a smaller diameter fiber at the distal end 242 of the tapered optical fiber 128 concentrates the projection of the laser beam from the distal tip 216 on a smaller surface area, thereby enabling smaller spot size cuts by the handpiece device 112 for higher precision. In some instances, the smaller diameter fiber at the distal end 242 of the tapered optical fiber 128 also enables projecting the laser beam with less energy, which may be beneficial for certain ophthalmic procedures.

The tapered optical fiber 128 may be configured to operate as an optical waveguide to propagate the laser light 241. The tapered optical fiber 128 may be configured to project the laser light 241 from the distal end 242 of the tapered optical fiber 128. In other embodiments, the characteristics of the laser light 241 propagated through the tapered optical fiber 128 are such that the laser light 241 causes disruption of the vitreous collagen fibers within the path of the laser light 241. Disruption refers to the breaking down of the tissue by rapid ionization of molecules thereof. In certain embodiments, the laser light 241 propagated by the tapered optical fiber 128 has a wavelength between about 2.0 μm and about 3.5 μm, such as between about 2.5 μm and about 3.3 μm. However, the techniques disclosed herein may implement any suitable type of laser light for ophthalmic surgery.

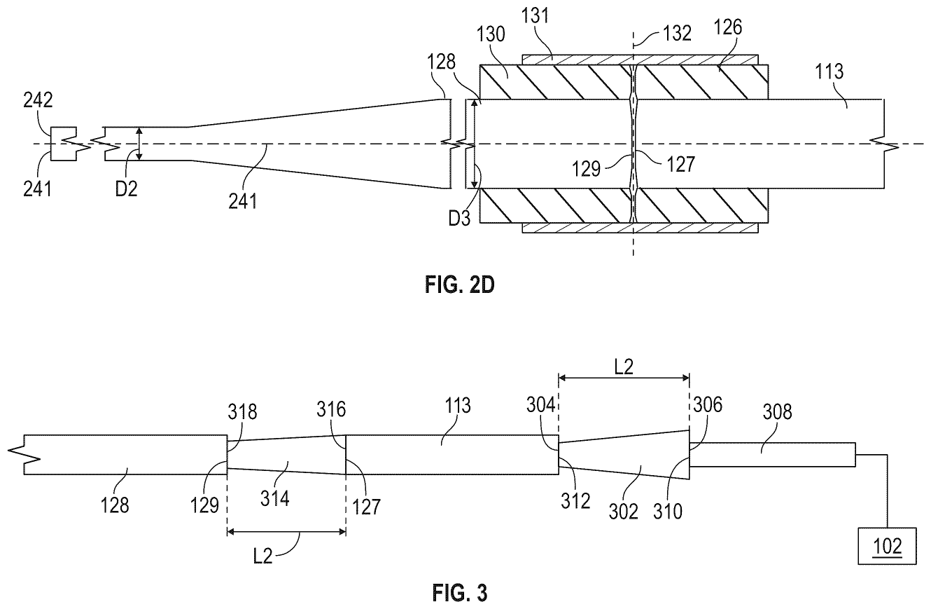

FIG. 3 illustrates a plan view of exemplary tapered optical fibers used to couple the optical fiber 113 of the cable assembly 111 with adjacent optical fibers, according to certain embodiments of the present disclosure. In some embodiments, a distal end 306 of a delivery fiber 308 from the laser system 102 is configured to focus the laser light 241 being delivered on a core of the optical fiber 113 to transmit the laser light 241 to the tapered optical fiber 128 through the optical fiber 113. As discussed above, in some instances, laser light transmitted between coupled ends of adjacent optical fibers may be partially delivered to the adhesive surrounding the core of the optical fiber receiving the laser light, such as optical fiber 113 receiving laser light from the delivery fiber 308 in the example shown. Such heating of the adhesive may damage or cause the optical fiber 113 to fail.

As shown in FIG. 3, in some embodiments, a first tapered optical fiber segment 302 having a tapering shape similar to the tapered optical fiber 128 may therefore be implemented at the coupling interface between a proximal end 304 of the optical fiber 113 and the distal end 306 of the delivery fiber 308. The first tapered optical fiber segment 302 can assist in concentrating the delivery of the energy of the laser light 241 on a smaller area aligned with the core of the optical fiber 113. Coupling the optical fiber 113 and the delivery fiber 308 of the laser system 102 with the first tapered optical fiber segment 302 decreases the likelihood of misalignment (and the resulting heating) between the delivery fiber 308 and the adhesive of the optical fiber 113 at the proximal end 304.

To limit the potential for damage to the first tapered optical fiber segment 302 itself, the first tapered optical fiber segment 302 includes a proximal end 310 having a diameter greater than a diameter of the distal end 306 of the delivery fiber 308. In some embodiments, when coupling the proximal end 310 of the first tapered optical fiber segment 302 with the distal end 306 of the delivery fiber 308 having a diameter of about 200 μm, the proximal end 310 of the first tapered optical fiber segment 302 may have a diameter between about 200 μm and 300 μm, such as between about 230 μm and about 270 μm, such as about 250 μm, but may have a larger or smaller diameter in some embodiments. Implementing the proximal end 310 of the first tapered optical fiber segment 302 with a larger diameter limits the chances of the first tapered optical fiber segment 302 itself being damaged by the errant energy of the laser light 241 delivered by the delivery fiber 308.

As with the tapered optical fiber 128, the diameter of the first tapered optical fiber segment 302 may similarly decrease towards a distal end 312 of the first tapered optical fiber segment 302 to concentrate and deliver the laser light 241 to a smaller area. In some embodiments, when coupling the proximal end 304 of the optical fiber 113 having a diameter of about 200 μm at the core (e.g., about 230 μm including cladding) with the distal end 312 of the first tapered optical fiber segment 302, the distal end 312 may in turn have a diameter between about 150 μm and 230 μm, such as between about 160 μm and about 200 μm, such as about 180 μm, but may have a larger or smaller diameter in some embodiments.

The optical fiber 113 further include a second tapered optical fiber segment 314 disposed between the distal end 127 of the optical fiber 113 and the proximal end 129 of the tapered optical fiber 128 to assist in the delivery of the laser light 241 to the tapered optical fiber 128 thorough the optical fiber 113. In some embodiments, when coupling the distal end 127 of the optical fiber 113 having a diameter of about 200 um at the core (about 230 um including cladding) with a proximal end 316 of the second tapered optical fiber segment 314, the proximal end 316 of the second tapered optical fiber segment 314 may have a diameter between about 180 μm and 260 μm, such as between about 200 μm and about 240 μm, such as about 220 μm, but may have a larger or smaller diameter in some embodiments.

As with the first tapered optical fiber segment 302, the diameter of the second tapered optical fiber segment 314 similarly decreases towards a distal end 318 of the second tapered optical fiber segment 314 to concentrate the delivery of the laser light 241. In some embodiments, when coupling the proximal end 129 of the tapered optical fiber 128 having a diameter of about 200 um with the distal end 318 of the second tapered optical fiber segment 314, the distal end 312 of the second tapered optical fiber segment 314 may have a diameter between about 110 μm and 200 μm, such as between about 130 μm and about 180 μm, such as about 150 μm, but may have a larger or smaller diameter in some embodiments.

In some embodiments, the first and second tapered optical fiber segments 302, 314 may have a length L2 between 8 mm and 20 mm, such as between about 10 mm and 15 mm, such as about 12 mm, but may have a larger or smaller length in some embodiments. In some embodiments, the first and second tapered optical fiber segments 302, 314 may include a single-crystal optical fiber similar to the tapered optical fiber 128 discussed above (e.g., sapphire core fiber), while the optical fiber 113 may include an optical fiber with a core and a cladding, such as a germanium-oxide glass optical fiber. Using a germanium-oxide glass optical fiber for the optical fiber 113 enables the cable assembly 111 (and the optical fiber 113 disposed therein) to be more flexible than a corresponding sapphire core fiber having the same diameter.

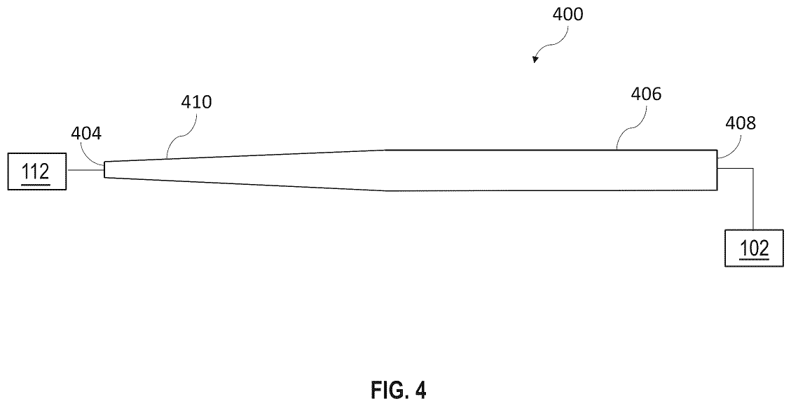

FIG. 4 illustrates an exemplary optical fiber that may be implemented in the surgical system 100 described in FIG. 1A, according to certain embodiments of the present disclosure. Even with the use of the first and second tapered optical fiber segments 302, 314 to assist with alignment between coupled optical fibers, some amount of optical loss may nonetheless occur at the coupling interfaces between the optical fiber 113 and each of the first and second tapered optical fiber segments 302, 314. Optical loss may also be experienced at the coupling interface between the optical fiber 113 and the tapered optical fiber 128 in the handpiece device 112 discussed above. As shown in FIG. 4, in further embodiments, the optical fiber 113 and the tapered optical fiber 128 may be formed as a single unitary optical fiber 400 extending between the laser system 102 of surgical system 100 and the probe 110 of the handpiece device 112 for propagating the laser light 241 and without the need for coupling of any optical fibers.

In some embodiments, the optical fiber 400 includes a proximal portion 406 coupled at a proximal end 408 to the laser system 102, and a distal portion 410 extending into the handpiece device 112 and terminating at a distal end 404 within the probe 110 near the distal tip 216. In some embodiments, the optical fiber 400 has a length between about 1 meter and about 3 meters, such as about 2 meters, but may have a larger or smaller length in some embodiments. Similar to the tapered optical fiber 128, the optical fiber 400 may comprise a single-crystal optical fiber, such as a tapered sapphire core fiber disposed in a cable jacket. The optical fiber 400 may alternatively include an optical fiber made of any of the other material described above for the tapered optical fiber 128.

As described above, sapphire core fibers having diameters below 200 um may exhibit excessive transmission loss. However, sapphire core fibers with diameters of 200 um or more are relatively stiff and may present ergonomic issues for the user/surgeon using the handpiece device 112 connected to the distal end 404 of the optical fiber 113. As with the tapered optical fiber 128, the sapphire core fiber in the optical fiber 400 provides the optical fiber 400 with a tapered shape. To improve transmission through the optical fiber 400, the optical fiber 400 may in turn include a tapered shape resulting in the proximal portion 406 of the optical fiber 400 initially having a greater diameter to facilitate transmission of the laser light. The optical fiber 400 may then taper to a smaller diameter towards the distal portion 410 as the optical fiber 400 approaches the handpiece device 112 to provide the optical fiber 400 with improved flexibility proximate to the handpiece device 112.

In certain embodiments, the proximal portion 406 of the optical fiber 400 may have a diameter between about 250 μm and about 500 μm, such as between about 350 μm and about 450 μm, such as about 400 μm, but may have a larger or smaller diameter in some embodiments. In some embodiments, the distal portion 410 of the optical fiber 400 may have a diameter between about 50 μm and about 200 μm, such as between about 80 μm and 120 μm, such as about 100 μm, but may have a larger or smaller diameter in some embodiments. Similar to the tapered optical fiber 128, the tapered shape enables the optical fiber 400 to have a sufficiently small diameter near the distal end 404 disposed in the probe 110 of the handpiece device 112 and may provide similar advantages as described above.

In summary, embodiments of the present disclosure include devices and systems for use in performing laser assisted ophthalmic surgeries. In particular, the handpiece device and system described above may include projecting laser light from a tapered optical fiber (e.g., sapphire core fiber, made of a sapphire material) partially disposed within the probe of the handpiece device, thereby enabling improved transmission characteristics of laser light through probes with smaller gauges. In some embodiments, utilization of the tapered optical fiber in a laser vitrectomy probe for severing collagen fibers of vitreous materials also allows the probe to maintain adequate aspirating functions to simultaneously remove the severed vitreous material as the surgery is performed. Furthermore, utilization of the tapered optical fiber at the optical interfaces between the optical fiber of the cable assembly and the laser system (or laser source therein) and/or the handpiece device reduces the risk of damage to the cable assembly and may increase the useful life of the cable assembly. In some embodiments, the optical fiber of the cable assembly (e.g., a hollow core fiber or made from germanate or fluoride glass) is coupled to the tapered optical fiber to efficiently deliver laser light to the tapered optical fiber in the handpiece device. Still further, some of the embodiments described herein improve the transmission of laser light to the handpiece device through sturdier single-crystal optical fibers that are easier to work with, while still maintaining sufficient ergonomics for the handpiece device connected thereto to be manipulated by the user. Accordingly, the described embodiments enable the performance of more efficient, less invasive, and safer ophthalmic procedures.

Although vitreous and phacoemulsification surgeries are discussed as examples of a surgical procedure that may benefit from the described embodiments, the advantages of the surgical devices and systems described herein may benefit other ophthalmic procedures having light emitting functionality (e.g., laser light, illumination light, etc.).

While the foregoing is directed to embodiments of the present disclosure, other and further embodiments of the disclosure may be devised without departing from the basic scope thereof, and the scope thereof is determined by the claims that follow.

What is claimed is:

1. A handpiece device, comprising:
a housing;
a cable assembly disposed within the housing and extending from a supply port at a proximal end of the housing, the cable assembly comprising an optical fiber; and
a tapered optical fiber disposed in the housing and having a diameter that decreases between a proximal end of the tapered optical fiber and a distal end of the tapered optical fiber,
wherein the proximal end of the tapered optical fiber is optically coupled to a distal end of the optical fiber.

2. The handpiece device of claim 1, further comprising a probe disposed through an opening in a distal end of the housing, the probe having a lumen extending therethrough, wherein the distal end of the tapered optical fiber is disposed in the lumen.

3. The handpiece device of claim 2, wherein the lumen further comprises an annular gap circumferentially surrounding the tapered optical fiber, and the annular gap is configured to provide a coaxial path for aspiration through the lumen of the probe.

4. The handpiece device of claim 1, wherein a diameter of the distal end of the tapered optical fiber is between about 50 μm and about 150 μm.

5. The handpiece device of claim 1, wherein a diameter of the proximal end of the tapered optical fiber is between about 180 μm and about 250 μm.

6. The handpiece device of claim 1, wherein the optical fiber of the cable assembly comprises a diameter between about 180 μm and about 400 μm.

7. The handpiece device of claim 1, wherein the tapered optical fiber comprises a single-crystal fiber composed of a material comprising α-Al₂O₃ (Sapphire), Ti:Sapphire, Y₃Al₅O₁₂ (YAG), Ho:YAG, Yb:YAG, Nd:YAG, Er:YAG, Ce:YAG, Cr:YAG, or ZrF₄—BaF₂—LaF₃—AlF₃—NaF (ZBLAN).

8. A cable assembly for a surgical system, comprising:
an optical fiber having a proximal end and a distal end;
a first tapered optical fiber having a distal end and a proximal end, the distal end of the first tapered optical fiber optically coupled to the proximal end of the optical fiber, wherein:
the first tapered optical fiber comprises a first diameter that decreases from the proximal end of the first tapered optical fiber to the distal end of the first tapered optical fiber, and
a diameter of the distal end of the first tapered optical fiber is less than a diameter of the proximal end of the optical fiber; and
a second tapered optical fiber, wherein a proximal end of the second tapered optical fiber is optically coupled to the distal end of the optical fiber.

9. The cable assembly of claim 8, wherein the second tapered optical fiber comprises a second diameter decreasing from the proximal end of the second tapered optical fiber to a distal end of the second tapered optical fiber, and a diameter of the proximal end of the second tapered optical fiber is greater than a diameter of the distal end of the optical fiber.

10. The cable assembly of claim 8, wherein a distal end of the second tapered optical fiber is configured to optically couple with a proximal end of a third optical fiber disposed in a handpiece device, wherein a diameter of the proximal end of the third optical fiber is greater than or equal to a diameter of the distal end of the second tapered optical fiber.

11. The cable assembly of claim 8, wherein the proximal end of the first tapered optical fiber is configured to optically couple with a laser system.

12. The cable assembly of claim 11, wherein at least one of the first tapered optical fiber and the second tapered optical fiber comprises a single-crystal fiber composed of a material comprising α-Al2O3 (Sapphire), Ti:Sapphire, Y3Al5O12 (YAG), Ho:YAG, Yb:YAG, Nd:YAG, Er:YAG, Ce:YAG, Cr:YAG, or ZrF4—BaF2—LaF3—AlF3—NaF (ZBLAN).

13. A surgical system, comprising:
a surgical console comprising a laser system; and
a handpiece device comprising:
a housing;
a cable assembly disposed within the housing and extending from a supply port at a proximal end of the housing, the cable assembly comprising an optical fiber having a proximal end and a distal end; and
a tapered optical fiber disposed in the housing and having a diameter that decreases between a proximal end of the tapered optical fiber and a distal end of the tapered optical fiber,
wherein the proximal end of the optical fiber is optically coupled with the laser system, and the distal end of the optical fiber is optically coupled to the proximal end of the tapered optical fiber.

14. The surgical system of claim 13, wherein:
the surgical console further comprises an aspiration system;
the handpiece device further comprises a probe disposed through an opening in a distal end of the housing, the probe having a lumen extending therethrough, and the distal end of the tapered optical fiber being disposed in the lumen; and
the lumen is in fluid communication with the aspiration system and further comprises an annular gap circumferentially surrounding the tapered optical fiber, the annular gap being configured to provide a coaxial path for aspiration through the lumen of the probe.

15. The surgical system of claim 13, wherein the tapered optical fiber comprises a single-crystal fiber composed of a material comprising α-Al2O3 (Sapphire), Ti:Sapphire, Y3A15O12 (YAG), Ho:YAG, Yb:YAG, Nd:YAG, Er:YAG, Ce:YAG, Cr:YAG, or ZrF4—BaF2—LaF3—AlF3—NaF (ZBLAN).
